(19) United States
(12) Patent Application Publication
(10) Pub. No.: US 2026/0015615 A1
(43) Pub. Date: Jan. 15, 2026

(54) SPERMATOGENESIS ALTERATION

(71) Applicant: University of Virginia Patent Foundation, Charlottesville, VA (US)

(72) Inventors: Seham Ebrahim, Charlottesville, VA (US); Wenxuan Zheng, Charlottesville, VA (US)

(21) Appl. No.: 19/269,522

(22) Filed: Jul. 15, 2025

Related U.S. Application Data
(60) Provisional application No. 63/671,569, filed on Jul. 15, 2024.

(51) Int. Cl. C12N 15/113; A61P 15/08

(52) U.S. Cl. CPC C12N 15/113; A61P 15/08; C12N 2310/14

(57) ABSTRACT

A method and composition for modulating male fertility are disclosed, centered on the targeted alteration of transmembrane channel-like (TMC) protein function, activity, or gene expression. Small molecules or pharmaceutically acceptable salts or solvates thereof are provided to modulate the activity or expression of TMC proteins, such as TMC5, in testis tissue. The compositions may include TMC5 channel blockers, dimerization inhibitors, TMC5-CIB1 interaction inhibitors, or small interfering RNAs targeting TMC5 mRNA, optionally formulated with a pharmaceutically acceptable carrier and capable of crossing the blood-testis barrier. Methods of diagnosing male infertility are also provided, comprising quantitating TMC5 expression or activity and comparing to a standard to identify infertility. These approaches enable reversible, non-hormonal regulation of spermatogenesis and fertility, offering new therapeutic strategies for male contraception and infertility treatment.

### TECHNICAL FIELD

Embodiments disclosed herein generally relate to the field of reproductive regulation technology; and to the alteration of spermatogenesis with subsequent impact upon fertility.

### BACKGROUND

Infertility affects ˜10% of men of reproductive age. The etiology of idiopathic male infertility remains largely unknown, presenting a significant ongoing challenge for biomedical science. Azoospermia, characterized by the complete absence of sperm in ejaculate, represents the most severe form of male infertility. Despite its extreme phenotype, azoospermia is remarkably prevalent, affecting 10-20% of infertile men. While some azoospermia cases result from physical blockages in the genital tract, the majority are classified as non-obstructive azoospermia (NOA), caused by impaired sperm development. NOA is of additional interest due to its association with higher rates of testicular cancer and increased risk of death. However, the understanding of the monogenic causes of the condition is severely limited, with no “recurrently” mutated NOA gene identified to date. Identifying single genes associated with NOA is crucial for discovering new targets for male infertility treatment and developing male contraceptives, for which there is ever increasing demand and only few viable candidates.

Thus, there remains a need in the art for male contraceptives that do not rely upon anatomic obstruction.

### SUMMARY

Embodiments disclosed herein relate to the transmembrane channel-like (TMC) protein family. In particular embodiments, downregulation/ablation of the expression of a gene within the TMC family leads to male infertility. In certain embodiments, the infertility occurs without any other associated phenotypes. In certain embodiments the affected gene is TMC5.

In still other embodiments, the upregulation of the expression of a gene within the TMC family restores fertility. In still other embodiments, a TMC protein complex is provided or repaired.

In still other embodiments one or more small molecules may modulate a TMC family protein, altering a fertile/infertile state and/or increasing/decreasing the activity of a TMC family protein.

The present disclosure provides compositions and methods for modulating male fertility by targeting transmembrane channel-like (TMC) proteins, particularly TMC5, in testis tissue. In certain embodiments, the compositions comprise a small molecule or a pharmaceutically acceptable salt or solvate thereof that alters the activity or gene expression of a TMC protein, such as TMC5. The small molecule may function as a TMC5 channel blocker, a TMC5 dimerization inhibitor, a TMC5-CIB1 interaction inhibitor, or a small interfering RNA targeting TMC5 mRNA, and may be formulated with a pharmaceutically acceptable carrier and designed to cross the blood-testis barrier.

The disclosure further provides methods for diagnosing male infertility by quantitating the expression level or activity of TMC5 in a biological sample, comparing the result to a standard expression level, and diagnosing infertility when the measured level is below the standard. The quantitation may be performed on semen, testicular tissue, or blood plasma, and may involve nucleic acid amplification assays or measurement of calcium influx in isolated spermatogenic cells. The diagnostic method may further include correlating TMC5 expression with semen quality parameters and repeating the assessment after administration of a TMC5-modulating agent. Additionally, methods are provided for decreasing male fertility by administering to a subject a composition that downregulates TMC protein expression in testis tissue. These approaches enable reversible, non-hormonal regulation of spermatogenesis and fertility, supporting new strategies for male contraception and infertility treatment.

These and other features and characteristics are more particularly described below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. Features and advantages of embodiments of the present invention will become apparent on reading the detailed description below with reference to the drawings, which are illustrative but non-limiting, wherein:

FIG. 1 illustrates the expression of TMC5 RNA is enriched in spermatids in mice (FIG. 1, a) and humans (FIG. 1, b).

FIG. 2 presents fluorescent micrograph results showing spermatid elongation in mice is blocked in the absence of TMC5.

FIG. 3 illustrates the localization of TMC5 to the plasma membrane of spermatids.

FIG. 4 Illustrates the impairment of calcium influx in response to membrane tension changes in the absence of TMC5. FIG. 4A presents calcium influx in a wildtype mouse. FIG. 4B presents calcium influx in a knock-out mouse.

FIG. 5 illustrates TMC5 and CIB1 interactions in spermatogenesis.

FIG. 6 illustrates the TMC5-CIB1 calcium-ion complex in silico model along with multiple small molecule target routes.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

### Abbreviations:

AnV Annexin V
KO knockout
MET mechano-electrical transducer
NOA non-obstructive azoospermia
PS phosphatidylserine
RB residual body
TMC transmembrane channel-like
WT wildtype

Several challenges exist in identifying single genes linked to azoospermia. Sperm production involves the coordinated action of numerous genes, many of which are predicted to interact with each other. Moreover, the nature of infertility makes it difficult to implement standard approaches for identifying disease-associated genes in human populations, such as genome-wide family-based studies.

Transmembrane channel-like (TMC) proteins are highly conserved mechanosensitive membrane proteins, encompassing eight family members (TMC1-TMC8) identified across species from mammals like humans and mice to C. elegans. These proteins typically feature ten transmembrane domains and are predicted to form dimers. TMC1 and TMC2 are recognized as mechano-electrical transducer (MET) channels in cochlear hair cells. Mutations in these proteins or their binding partners disrupt hair cell mechanoelectrical transduction, leading to deafness in humans and mice. TMC gene therapy can restore auditory function and balance in mice. CryoEM studies of C. elegans TMC1/2 reveal that these proteins form dimers and are complexed with other proteins like C. elegans CIB1 and TMIE. Functional characterizations suggest that TMC1 regulates membrane homeostasis, as depletion leads to disrupted phosphatidylserine (PS) externalization under stimulation. While TMC1 and TMC2 have been extensively studied due to their roles in hearing conduction, the full spectrum of functional roles across the TMC family remains underexplored. Despite considerable advances, direct evidence of the structure or molecular function of mammalian TMCs remains elusive, primarily due to challenges in isolating TMC1 complexes from vertebrate sources and the ineffectiveness of producing functional complexes through recombinant methods in heterologous cell lines.

This disclosure focuses on TMC5, abundantly expressed in the gut and testis, leveraging tissues with ample material for research. Previous spatiotemporal analysis using Tmc5-mCherry mice revealed dynamic expression at the enterocyte microvilli tips in the postnatal mouse gut, where the glycocalyx filaments are subject to variable forces. Predictions from AlphaFold indicate high structural similarity between TMC1 and TMC5, including conserved residues, suggesting similar functions in ion and membrane homeostasis. Molecular dynamics simulations of homology-based structural models demonstrated that while the TMC5 pore permits ion permeability, its high lipid permeability often impedes ion passage, particularly in the presence of the Cib3 protein. These observations have steered current research directions, positing TMC5 as a multi-protein lipid scramblase, pivotal for understanding its physiological and molecular functions.

This disclosure presents that Tmc5 is highly expressed in spermatids and plays a crucial role in spermiogenesis by regulating ion/membrane homeostasis. Spermiogenesis is the post-meiotic phase of male germ cell development during which the haploid spermatids differentiate into mature spermatozoa. Mouse spermiogenesis consists of sixteen (16) steps characterized by morphological changes in acrosome formation, nuclear condensation and elongation, flagellum formation, and cytoplasm removal whose molecular underpinnings remain poorly understood. Maintaining cellular homeostasis is essential for the spermatids to go through dramatic morphological change. Spermatid calcium homeostasis is highly regulated, as spontaneous calcium influx can be observed in the spermatids, and the concentration of spermatids significantly increases as it elongates and condensates. Spermatid membrane homeostasis is crucial as lacking highly unsaturated fatty acids causes failure of late spermiogenesis. Male mice lacking lipid scrambling mediated by X-linked XK blood group-related 8 (Xkr8) are infertile due to reduced sperm count in the epididymis.

The localization and interaction of the Tmc5 protein with its binding partner, Cib1, at the spermatid membrane was studied using Tmc5-mCherry mice. To demonstrate that Tmc5 is vital for normal spermiogenesis by regulating membrane (and calcium) homeostasis Tmc5 KO mice were created. The absence of Tmc5 disrupts lipid scrambling, leading to the failure of RB detachment and subsequent cell death, with affected cells likely entirely phagocytosed by Sertoli cells. The findings disclosed herein provide new insights into membrane homeostasis during spermiogenesis and identify Tmc5 as a potential target for non-hormonal male contraceptives.

This research has direct implications for human health, as it provides valuable insights into a new cause of non-obstructive azoospermia (NOA), which affects 10-20% of infertile men. Furthermore, the classification of TMC5 as part of the ‘Druggable Proteome’-proteins that can be modulated by small molecules-highlights its potential as a therapeutic target. Remarkably, only 5-10% of these proteins are currently targeted by FDA-approved drugs, emphasizing the innovative and significant impact of targeting TMC5 in future therapeutic developments.

This disclosure provides pivotal insights into the molecular and physiological roles of TMC5 in spermatogenesis. This disclosure identifies TMC5 as a critical component of a multi-protein membrane complex essential for maintaining membrane homeostasis during normal spermiogenesis. A breakthrough of the research is the discovery of organized PS externalization during spermatid elongation, a process that resolves the longstanding question of how excess cytoplasm is eliminated during spermiogenesis. The disclosure demonstrates that the formation of the residual body (RB), characterized by externalized PS, likely facilitates its phagocytosis by Sertoli cells. Furthermore, this disclosure provides the first evidence that TMC5 may directly mediate PS externalization, potentially acting as a lipid channel. This novel insight into the role of TMC5 in PS externalization not only highlights its critical function in spermatid development but also suggests that TMC1 and other members of the TMC family may have similar roles, broadening our perspective on how ion channels and lipid scramblases contribute to cellular integrity and function. This knowledge opens up potential therapeutic strategies, including the development of novel non-hormonal male contraceptives.

Data, below further described and presented, indicate a complete absence of elongated spermatids in the testes of these mice, making this a compelling new model for monogenic NOA. Using super-resolution microscopy, we determined that TMC5 is highly expressed in heads of mature spermatids in healthy mice. This is consistent with the enrichment of TMC5 in human spermatids and reported links to male infertility in humans, speaking to the direct translatability of the role of TMC5 from mouse to man. Additionally exciting is that TMC5 is an ion channel and part of the Druggable Proteome, proteins that can be modulated by small molecules, of which only 5-10% are currently targeted by FDA-approved drugs. TMC5 is thus an attractive target for developing therapies for male infertility and/or male contraceptives.

Presented below and further described, mRNA sequencing data revealed that Tmc5 is consistently enriched throughout the stages of spermatid development, a finding that was corroborated by Tmc5-mCherry knock-in mice. The knock-in mice demonstrated Tmc5 enrichment at all stages of spermatid development. Spatiotemporal characterization of Tmc5-mCherry in spermatogenic cells highlights the complex role played by the protein, particularly noting its localization at the spermatid membrane and significant enrichment in elongating spermatids and the residual body. This specific localization suggests that Tmc5 plays a critical role in cytoplasmic elimination and midpiece formation. Additionally, Tmc5 localization persists in spermatozoa within the epididymis, where it predominantly marks the midpiece and cytoplasmic droplet, indicating its continued importance post-testis.

The expression of Tmc5 in the testis opens a novel avenue for exploring the molecular functions of Tmc5. Utilizing Tmc5-mCherry mice, we successfully isolated the Tmc5-mCherry complex, including Cib1. Evidence from various in vivo and in vitro methods, including experiments with heterologous cell lines, supports the interaction between Tmc5 and Cib1.

This disclosure is the first to demonstrate that normal spermiogenesis requires organized phosphatidylserine (PS) externalization, likely facilitated by members of the TMC family, namely Tmc5 Their coordinated action is crucial for maintaining membrane homeostasis during spermiogenesis, particularly for PS externalization which signals phagocytosis by Sertoli cells. The precise co-localization of Tmc5 and Tmc1 with the externalized PS strongly suggests that they directly mediate this process. Furthermore, the observed dysregulation of PS externalization and Tmc1 in the absence of Tmc5 underscores the direct role of Tmc5 in facilitating PS externalization.

Additionally, while it is known that significant force is applied during spermatid elongation to accommodate dramatic morphological changes, the mechanisms by which these forces are generated remain poorly understood. Without subscription or binding to a particular theory or mechanism of operation, the disclosed findings suggest that the coordinated action of Tmc5 and Tmc1 in PS externalization may be involved in force generation.

In summary, this disclosure not only sheds light on the functional dynamics of Tmc5 in spermiogenesis but also sets the stage for further investigations into the biophysical aspects of sperm cell development.

The deletion of Tmc5 resulted in failed spermiogenesis, evidenced by reduced testis weight and an absence of mature sperm in the epididymis. This phenotype underscores Tmc5's crucial role in spermatogenesis. Specifically, Tmc5 depletion led to cell death during spermiogenesis, failed cytoplasm elimination, defective midpiece formation, and disrupted tail formation, confirming our hypotheses based on Tmc5-mCherry localization studies. Additionally, our data suggest involvement of TMC5 in actin regulation, possibly through interactions with Espin, an actin-binding protein. This is analogous to the known role of TMC1 in actin regulation via Espin in the inner ear.

Collectively, these findings underscore the essential function of Tmc5 in the normal progression and completion of spermiogenesis, highlighting its pivotal role in the maturation of spermatids. The multifaceted roles of Tmc5 revealed by this disclosure not only deepen our understanding of spermatogenesis but also underscore the potential therapeutic targets within this pathway for treating male infertility.

### Results

### Expression and Localization of TMC5 During Spermatogenesis

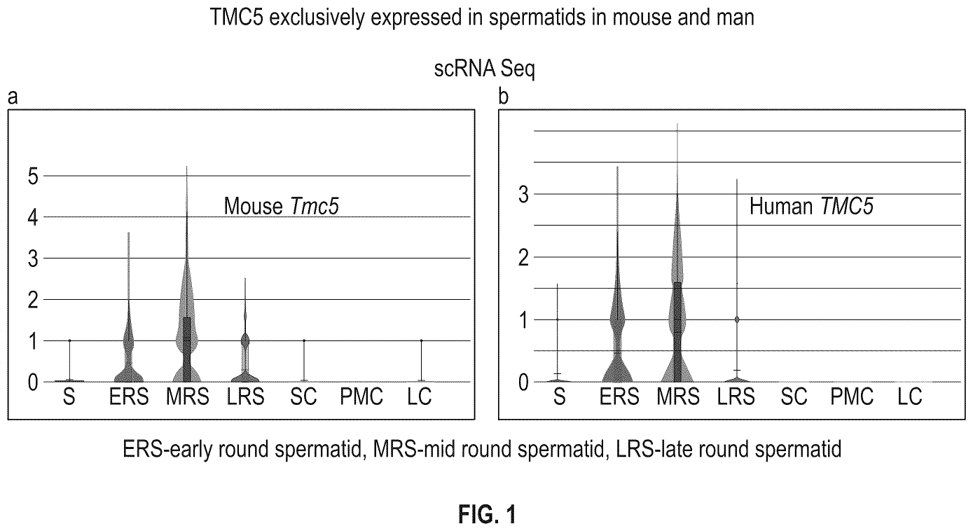

To investigate the role of TMC5 in spermatogenesis, we assessed its expression and localization in various stages of spermatogenic cells. Through single-cell mRNA analysis in adult mouse testis, we detected significant enrichment of Tmc5 expression in early (ERS), mid (MRS), and late (LRS) mouse (left) and human (right) spermatids (FIG. 1).

### TMC5 is Required for Normal Spermiogenesis

We created Tmc5 knockout (KO) mice by using the CRISPR/Cas9 system. Confirmation of Tmc5 KO was established through genetic verification. Phenotypic analysis revealed that while Tmc5 KO mice were viable, appeared normal, and maintained a typical metabolic rate, male KO mice exhibited smaller testes and a reduced testis/body weight ratio compared to WT mice (data not shown).

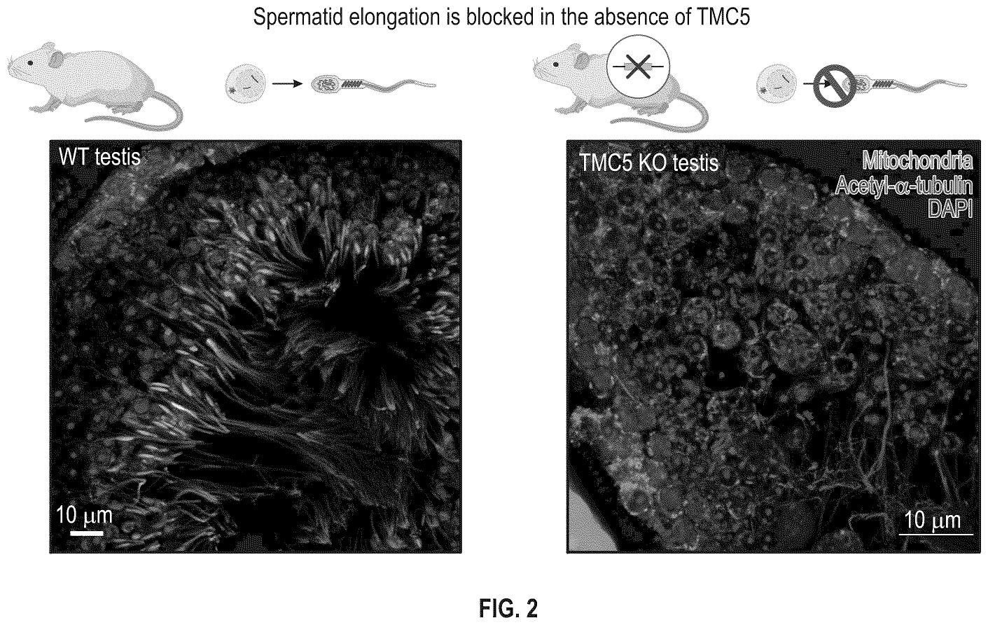

Importantly, as seen in FIG. 2, Tmc5 KO males displayed non-obstructive azoospermia, with no elongated spermatozoa reaching the caput epididymis, highlighting Tmc5's critical role in spermatogenesis and male fertility (right micrograph). Further investigations using immunofluorescence microscopy on the cryosectioned seminiferous tubules of the testis revealed spermiogenesis defects in Tmc5 KO mice. These defects were characterized by TNP1-positive elongating spermatids forming spherical aggregates or presenting with a deformed shape (not shown). Additionally, there was notable cytoplasmic retention, absence of midpiece formation, and dysregulation of acetyl-α-tubulin (not shown). We also observed dysregulation in the expression of espin, an actin-binding protein crucial for proper spermatid development (not shown).

### TMC5 is Localized to the Plasma Membrane of Spermatids

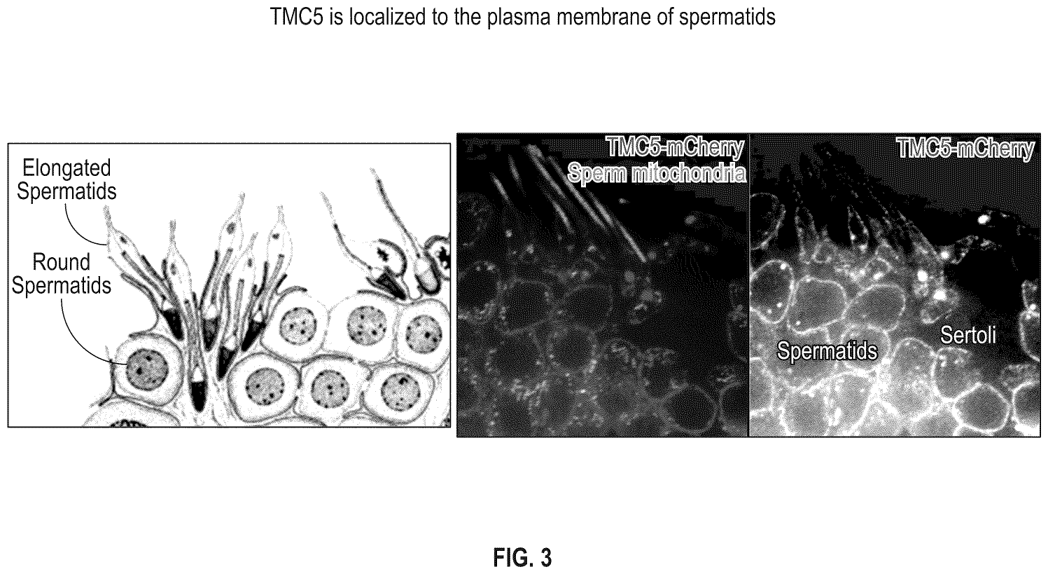

As seen in FIG. 3, TMC5 is localized to the plasma membrane of spermatids. FIG. 3, left, shows a micrograph containing round and elongated spermatids. The distinct expression pattern of Tmc5 in the testis was further explored using Tmc5-mCherry transgenic mice. Notably, mCherry fluorescence was mostly enriched on the membrane of spermatids (FIG. 3, center and right), but not in spermatogonia or spermatocytes, in cryosections showing Stage XII and VII tubules. The mCherry signal is retained in spermatozoa located in caput and cauda epididymis (not shown). Further detailed examination of Tmc5's precise membrane localization in spermatids and spermatozoa was conducted using live isolated spermatogenic cells from the testis and epididymis. We found that in early round spermatids, the mCherry signal was initially uniformly distributed, but as spermiogenesis progressed, it became increasingly enriched in the membrane of the cytoplasmic protrusion that eventually separates to form the RB (Not shown). In spermatozoa from the caput and cauda epididymis, the mCherry fluorescence was primarily localized to the mid-piece, including the cytoplasmic droplet and the head, highlighting a specific and sustained localization pattern (not shown). These findings highlight TMC5's crucial role in the development and maturation of male gametes, underscoring its importance in spermatogenesis and potential sperm functionality.

### Calcium Influx Altered in Response to TMC5 Presence

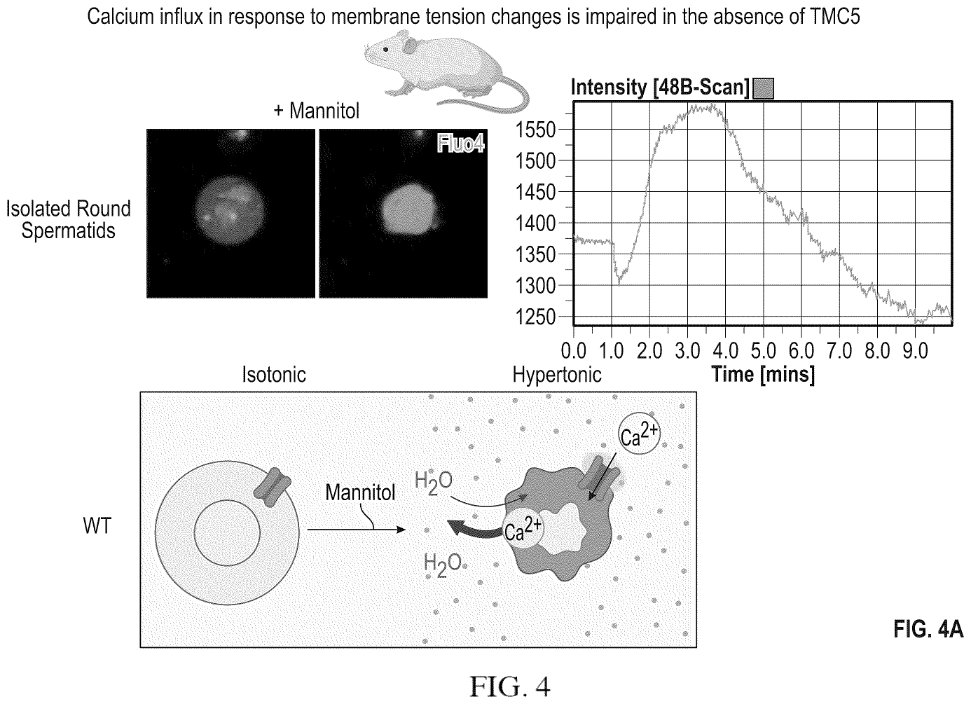

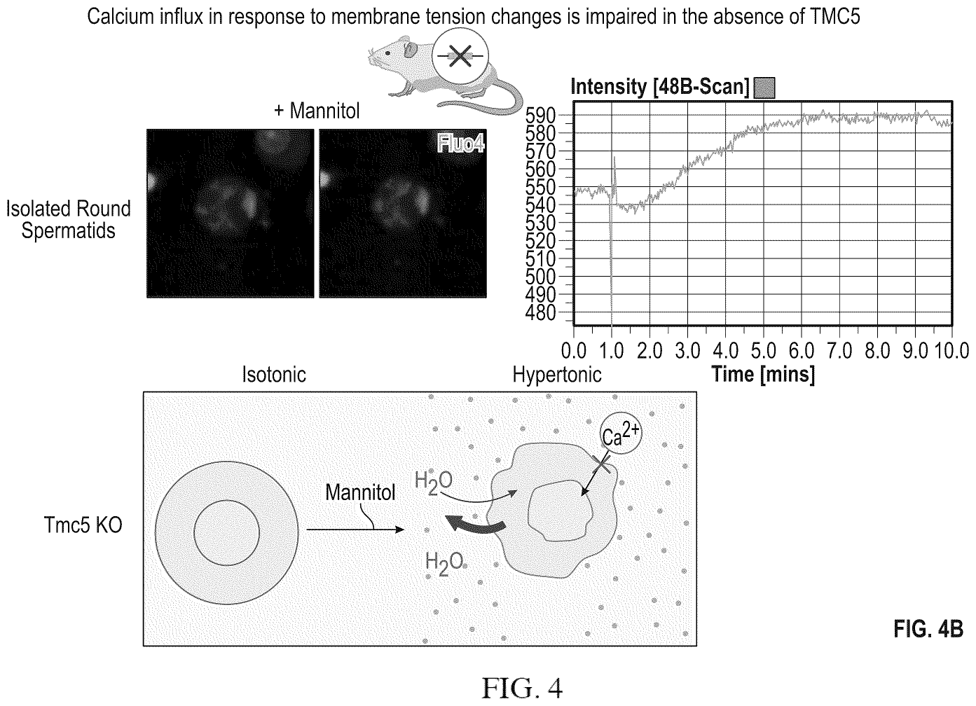

FIG. 4 Illustrates the impairment of calcium influx in response to membrane tension changes in the absence of TMC5. FIG. 4A presents calcium influx in a wildtype mouse. FIG. 4B presents calcium influx in a knock-out mouse. As seen in FIG. 4A, the addition of mannitol, altering the environment from isotonic to hypertonic results in the uptake of Fluo4 calcium indicator dye, as verified by the 48B-Scan intensity measurement. In contrast, as seen in FIG. 4B, no such change was seen in the KO mouse.

### TMC5 and CIB1 Interactions in Spermatogenesis and Cell Cultures

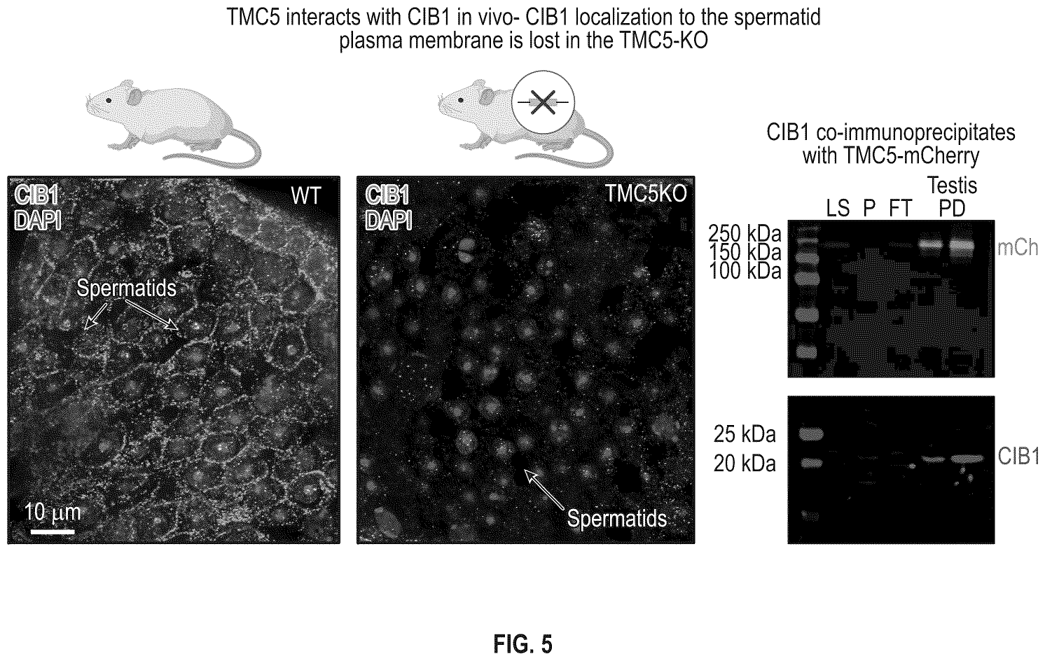

Previous studies have shown that members of the TMC family interact with the CIB family, with CIB1 being essential for spermatogenesis. To elucidate the biochemical interactions between TMC5 and CIB1, we conducted co-immunoprecipitation using Tmc5-mCherry testis lysate. Western blot analysis confirmed the presence of Tmc5-mCherry in the immunoprecipitates captured by the mCherry antibody, with concurrent co-immunoprecipitation of Cib1 (FIG. 5, right). Further, we assessed the impact of Tmc5 on Cib1 expression by comparing protein levels in WT and Tmc5 KO testis. The results indicated a reduction in Cib1 protein expression in the Tmc5 KO samples (not shown), supporting the interaction between Tmc5 and Cib1 proteins.

Using immunofluorescence microscopy, we validated the colocalization of Cib1 with Tmc5-mCherry at the spermatid membrane in WT mice (FIG. 5, left) and observed the loss of Cib1 targeting to the membrane in the Tmc5 KO mice (FIG. 5, center).

These in vivo findings prompted us to further explore the Tmc5-Cib1 interaction in vitro using SW1116 and COS7 cell cultures. SW1116 cells, which endogenously express Tmc5, demonstrated delivery of Tmc5-mCherry to the membrane at the tips of their microvilli, accompanied by an increase in Cib1 protein expression (not shown). Conversely, COS7 cells, which do not naturally express Tmc5, retain the Tmc5 protein in the ER and fail to transport it to the membrane. We co-transfected Tmc5 and Cib1 plasmid in COS7 cells and observed co-localization of Tmc5 and Cib1 (not shown). The presence of Tmc5 in the ER promotes the recruitment and binding of Cib1, while in its absence, Cib1 remains in the cytoplasm, indicating that Tmc5 is critical for the proper localization of Cib1.

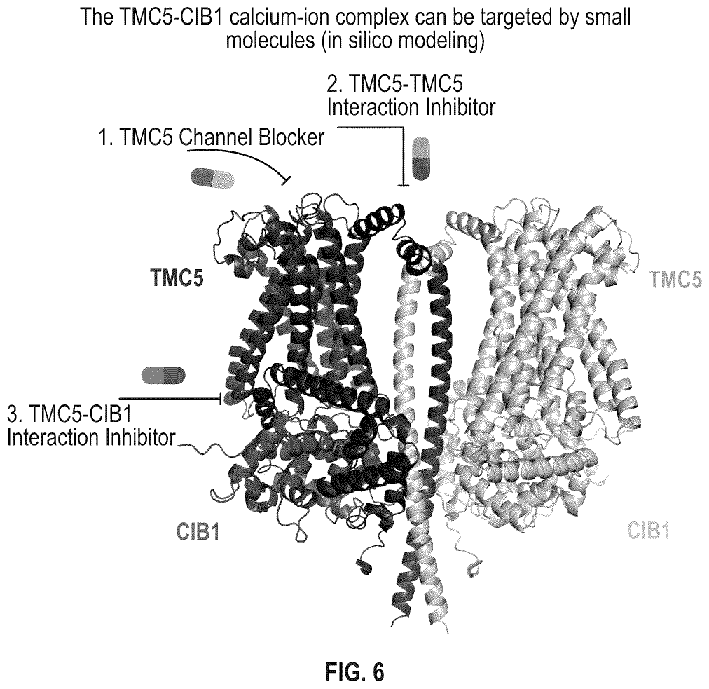

Finally, FIG. 6. illustrates the TMC5-C1B1 calcium-ion complex in silico model along with multiple small molecule target routes. As predicted by AlphaFold, and illustrated by the above data, TMC5-CIB1 form a calcium-ion complex that can be targeted by small molecules. Possible targets include: 1) TMC5 channel blockers; 2) TMC5-TMC5 interaction inhibitors; and, 3) TMC5-CIB1 interaction inhibitors.

### MATERIALS AND METHODS

### Antibodies and Other Staining Reagents

Suitable antibodies and staining reagents include: TMC1/5, CIB1/3, Actin, a-tub, TNP1, lectin PNA 488, Annexin V 488, concanavalin 647, PI, and all the secondary antibodies and staining reagents.

### Immunofluorescence and Light Microscopy

An adult male mouse was euthanized, and its testis was obtained, decapsulated, and fixed in 4% paraformaldehyde for 48 hours on a rotator at 4° C. Fixed testis was dehydrated with 15 min 10% and 20% sucrose solution incubation at room temperature followed by 30% sucrose solution incubation overnight at 4° C. Dehydrated testis was embedded in OCT, frozen on dry ice, and stored at −80° C. For microscopic analysis, 20 μm sections of the testis were cut using a cryostat, allowed to air-dry for 1 hour, and then washed in PBS for 10 minutes to remove OCT. The sections were permeabilized with 0.5% Triton X-100 for 45 minutes at room temperature. Antigen retrieval was performed by incubating the sections in a 10 mM sodium citrate buffer (pH 6.0) with 0.05% Tween-20 at 95-99.9° C. for 20 minutes. After cooling, the sections were blocked with 10% normal goat serum (NGS) overnight at 4° C. For immunofluorescence staining, the sections were incubated with primary antibodies diluted 1:400 in Can Get Signal Immunostain Solution A (code NKB-501) for 2 hours at room temperature or overnight at 4° C. Following primary incubation, the sections were washed three times in PBS and subsequently incubated with AlexaFluor 488/568/647-conjugated goat anti-rabbit, anti-rat, or anti-mouse secondary antibodies (Thermo Fisher), diluted 1:1000 in 10% NGS. After three further PBS washes, the sections were mounted using Fluoromount-G Mounting Medium containing DAPI (Thermo Fisher, catalog #00-4958-02). Confocal images were captured using an inverted fluorescence microscope (Nikon ECLIPSE Ti2) equipped with a spinning disk (YOKOGAWA CSU-WI confocal scanner unit). All image acquisition was managed through NIS-Element software (Nikon Instrument).

mCherry Pull-Down Assays

An entire testis from a TMC5-mCherry adult mouse, weighing approximately 100 mg, was flash frozen and subsequently stored at −80° C. The testis was then homogenized at 4° C. for 2 h in 400 μL glycol-diosgenin (GDN) lysis buffer containing 50 mM Tris-Cl (pH 8.8), 50 mM NaCl, 5 mM EDTA, 2% (w/v) GDN, and complete EDTA-free Protease Inhibitor Cocktail (Sigma, catalog #11836170001, with a ratio of 1 tablet per 10 mL of buffer). After centrifugation at 186,000 g for 50 min, the supernatant was transferred to a precooled new tube with an addition of 600 μL dilution buffer (lysis buffer without GDN), which was then applied to prewashed 50 μL ChromoTek RFP-Trap Magnetic Agarose (Proteintech, catalog #rtma) and incubated on a rotator at 4° C. for 2 h. The magnetic agarose was collected using a magnetic separation rack and washed 3 times with the dilution buffer, and then resuspended in a 160 μL 2×SDS sample buffer. To elute the TMC5-mCherry complex, the sample was boiled, and the magnetic agarose beads were subsequently removed via magnetic separation, isolating the target protein complex directly analyzed by SDS-PAGE and western blot.

### Western Blotting

One adult testis was homogenized at 4° C. for 2 h in 400 μL aforementioned GDN lysis buffer followed by centrifugation at 186,000 g for 50 min for membrane protein and RIPA lysis buffer (G-Biosciences catalog #786-489) with the Protease Inhibitor Cocktail for cytoplasmic protein followed by centrifugation at 14,000 g for 15 min. The resulting protein samples were prepared for gel electrophoresis by mixing them with an equal volume of 2× Laemmli Sample Buffer (Bio-rad, catalog #1610737) and boiling for 5 minutes. Protein concentrations were determined using a Bradford protein assay kit (Bio-rad, catalog #5000205), and 20-60 μg of protein was loaded into each lane of a 10-well SDS-PAGE gel. The protein sample was separated by SDS-PAGE and then transferred to a 0.22 μm nitrocellulose membrane (Bio-rad, catalog #1620097). The membrane was blocked in 5% skim milk overnight at 4° C., and then the membrane was washed three times in 0.1% TBST followed by incubation with primary antibody diluted 1:1000 in 5% skim milk for 2 h at room temperature. After three washes in 0.1% TBST, the membrane was incubated with secondary antibodies (Jackson ImmunoResearch Secondary Antibodies) for 45 min. Following a final series of three washes in 0.1% TBST, the membrane was imaged using a LI-COR Odyssey imaging system to visualize the protein bands.

### Spermatogenic Cells Isolation and AnV Assay

Two testes from one adult male mouse were obtained and decapsulated. Primary digestion was performed at 37° C. in 10 mL Digestion Media I containing 0.1 mL 50 mg/mL Collagenase A (Sigma, catalog #10103578001) and 0.1 mL 20 mg/mL DNase I (Sigma, catalog #10104159001) in 9.8 mL DMEM:F12 (Thermo Fisher, catalog #11320033). The digested testes were layered over the 40 mL 5% Percoll solution containing 4 mL 10×HBSS (Thermo Fisher, catalog #14065056), 2 ml Percoll (Sigma, catalog #P1644), and 34 mL ddH2O for 20 min at room temperature to allow highly dense seminiferous tubules to sink at the bottom. After discarding the top 35 mL supernatant, the bottom 5 mL was transferred to 10 mL Digestion Media II containing 2 mL 1× Trypsin, 0.1 mL 20 mg/mL DNase I, and 7.9 mL DMEM:F12 for secondary digestion for 20 min at 37° C. After pipetting the seminiferous tubules until they disappear, 3 mL FBS was added to neutralize the trypsin. The digested product was filtered through a 70 μm and 40 μm cell strainer (SPL Life Sciences, catalog #93070 and 93040), and then centrifuged at 500 g at 4° C. for 10 min. 1 mL of 1×RBC lysis buffer (Invitrogen, catalog #00-4300-54) was added to the cell pellet for 1 min at 4° C. immediately followed by centrifugation at 500 g at 4° C. for 2 min. Cell pellet was mixed with 1 mL DMEM:F12 for further analysis.

200 μL of the cell mixture was centrifuge at 500 g at 4° C. for 2 min. Cell pellet was incubated in 1 mL solution containing AnV-488 diluted at 1:1000, Hoechst-405 diluted at 1:1000 and 30 μL of PI in 1×HBSS with 2 mM calcium (Thermo Fisher, catalog #14025092) for 15 min at room temperature. After incubation, isolated cells were centrifuged at 500 g at 4° C. for 2 min, mixed with 2 mM calcium 1×HBSS, and then observed live using a 3.5 cm plate with glass slide bottom.

In general, the disclosure may alternately comprise, consist of, or consist essentially of, any appropriate components herein disclosed. The disclosure may additionally, or alternatively, be formulated so as to be devoid, or substantially free, of any components, materials, ingredients, adjuvants or species used in the prior art compositions or that are otherwise not necessary to the achievement of the function and/or objectives of the present disclosure. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this disclosure belongs.

The terms “fertility,” “fertility status,” “fertile,” “infertile,” and the like are used to either in specific, or in relative terms, describe the reproductive capacity of a subject as a whole, or for individual cells/gametes. Embodiments of the disclosure are believed to directly or indirectly alter fertility status either through alterations of gene expression (up or down regulated, blocked, etc.), provision of a missing protein or protein component, provision of one or more small molecules or biologics affecting a target protein or target protein gene (e.g., a “drug”).

Embodiments disclosed herein relate to the transmembrane channel-like (TMC) protein family. In particular embodiments, alteration/ablation/up or down regulation of the expression of a gene within the TMC family alters male fertility status. In certain embodiments, the infertility occurs without any other associated phenotypes. In certain embodiments the affected gene is TMC5. In additional embodiments, regulation of TMC protein activity, presence, or genetic composition alters a fertility status.

The disclosure includes a method of modulating prosome activity in a subject in need thereof, for example by administering an effective amount of an identified small molecule to the subject.

In some embodiments, an identified small molecule modulator or a pharmaceutically acceptable salt or solvate thereof is used to modulate the activity of one or more TMC proteins.

In some embodiments, the disclosure provides small molecules, e.g. a molecule of less than 1000 MW, that can target and regulate the production of one or more TMC or TMC complex constituent proteins by regulating, modulating, controlling the expression of, the methylation state of, or interfering with (e.g., RNAi, siRNA) the genomic sequence (DNA, RNA) encoding the proteins. In some embodiments host cells are harvested from a patient, treated with a small molecule to alter TMC structure or function and then returned to a patient.

Some embodiments relate to a method of modulating TMC activity in a subject in need thereof. In such embodiments an effective amount of an identified small molecule is administered to a subject in need thereof.

In some embodiments, an identified small molecule modulator or a pharmaceutically acceptable salt or solvate thereof may be used to modulate the activity of one or more TMC proteins.

In embodiments, such as the above, the fertility status of a subject is modulated by the treatment.

The terms “patient” or “subject” as used herein interchangeably and include humans and non-humans (e.g. companion animal). A patient can be a human patient having fertility issues related to over or under expression of one or more TMC proteins, in particular, TMC5.

An “effective dose,” “effective amount,” or “therapeutically effective amount” is an amount sufficient to produce the desired effect for which it administered, e.g., improvement in a fertility symptom or lessening in severity of a fertility symptom.) The exact amount of the effective dose will depend on the purpose of the treatment (e.g., restoration or cessation of male fertility as regulated by TMC5) and will be ascertainable by one of skill in the art using known techniques. In some embodiments and effective dose or effective amount is an amount sufficient to alter TMC5 activity, e.g. TMC5 activity in the testis, to at least 10%, 20%, 30%, 40%, 50%, 60%, 70%, 80%, or 90% of the median level of a patient of the same age that does not have a TMC5 alteration or to at least 10%, 20%, 30%, 40%, 50%, 60%, 70%, 80%, or 90% of the TMC5 activity level in a healthy control.

The terms “treat”, “treating” and “treatment” include alleviating, ameliorating, reducing the incidence, frequency or severity of, slowing or stopping the progress of, reversing or abrogating a medical condition or one or more symptoms or complications associated with the condition, and alleviating, ameliorating or eradicating one or more causes of the condition. Reference to “treatment” of a medical condition includes prevention of the condition. The terms “prevent”, “preventing” and “prevention” include precluding, reducing the risk or likelihood of developing, and delaying the onset of a medical condition or one or more symptoms or complications associated with the condition. In the context of TMC5 treatments this could be an increase or decrease in spermatogenesis depending on the desired outcome (e.g., increasing or decreasing male fertility).

The exact dosage for any treatment contemplated herein is chosen by an individual physician in view of a patient to be treated. Dosage and administration are adjusted to provide sufficient levels of embodiments of the disclosure to maintain the desired effect. Additional factors that may be taken into account include the severity of any disease state, age, weight, and gender of the patient; diet, time and frequency of the administration, drug combination(s), reaction sensitivities, and tolerance/response to therapy.

Short acting pharmaceutical compositions are administered daily whereas long-acting pharmaceutical compositions are administered every 2, 3 to 4 days, 1, 2, 3, 4, 5, or 6 weeks, or any range derivable therein, or every 1, 2, 3, 4, 5, 6, 7, 8, 9, 10, 11, 12 months, or more or any range derivable therein. Depending on half-life and clearance rate of the particular formulation, the pharmaceutical compositions of the disclosure may be administered once, twice, three, four, five, six, seven, eight, nine, ten or more times per day.

Normal dosage amounts for active ingredients may vary from approximately 1 to 100,000 micrograms, up to a total dose of about 10 grams, depending upon the route of administration. Desirable dosages include 250 μg, 500 μg, 1 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5 g, 6 g, 7 g, 8 g, 9 g, and 10 g.

More specifically, the dosage of the active ingredients described herein are those that provides sufficient quantity to attain a desirable effect, including those above-described effects. Accordingly, the dose of the active ingredients preferably produces a tissue or blood concentration of both about 1 to 800 μM. Preferable doses produces a tissue or blood concentration of greater than about 10 μM to about 500 μM. Preferable doses are, for example, the amount of active ingredients required to achieve a tissue or blood concentration or both of 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 110 μM, 120 μM, 130 μM, 140 μM, 150 μM, 160 μM, 170 μM, 180 μM, 190 μM, 200 μM, 220 μM, 240 μM, 250 μM, 260 μM, 280 μM, 300 μM, 320 μM, 340 μM, 360 μM, 380 μM, 400 μM, 420 μM, 440 μM, 460 μM, 480 μM, and 500 μM. Although doses that produce a tissue concentration greater than 800 μM are not necessarily preferred, they are envisioned and can be used with some embodiments of the present invention.

A constant infusion of embodiments of the invention can be provided so as to maintain a stable concentration of the therapeutic agents. The effective dose and method of administration of a particular embodiment of the instant invention may vary based on the individual patient and stage of any present diseases (e.g., fertility status), as well as other factors known to those of skill in the art.

Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosages for human use. The dosage of such compounds lies preferably within a range of circulating concentrations that include the ED50 with little or no toxicity. The dosage varies within this range depending upon the dosage form employed, sensitivity of the patient, and the route of administration

Pharmaceutical compositions of this disclosure can be formulated for administration by any route known to those of ordinary skill in the art that is suitable for porosome administration. Examples include intravenous, nasal, intradermal, intraarterial, intraperitoneal, intralesional, intracranial, intraarticular, intraprostatical, intrapleural, intratracheal, intravitreal, intravaginal, intrarectal, topical, intratumoral, intramuscular, subcutaneous, subconjunctival, intravesicular, mucosal, intrapericardial, intraumbilical, intraocularal, oral, topical, local, injection, infusion, continuous infusion, and localized perfusion administration, administration via a catheter, administration via a lavage, administration directly injected into the organ or portion of organ or diseased site of interest, or by other method or any combination of the forgoing as would be known to one of ordinary skill in the art. In a particular aspect, the composition can be formulated for nasal or oral inhalation or as a nebulized formulation. In some embodiments, the composition is a liquid. In other embodiments, the composition is a gel or a powder.

Pharmaceutical compositions of this disclosure will typically include a pharmaceutically acceptable carrier or excipient. The carrier is non-toxic, biocompatible, and does detrimentally affect the biological activity of the porosome or other active ingredients.

Pharmaceutical carriers or excipients suitable for inhaled or nebulized porosome compositions include diluents, stabilizers, propellants, surfactants, and preservatives. In some embodiments the pharmaceutical composition is a liquid inhaled or nebulized formulations and includes a diluent is selected from DMSO, ethylene glycol, glycerol, 2-Methyl-2,4-pentanediol (MPD), propylene glycol, sucrose, and trehalose. In some embodiments, the formulation comprises at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95%, diluent or any range discernable therein.

The pharmacologically active compounds of this invention can be processed in accordance with conventional methods of pharmacy and good manufacturing practices to produce medicinal agents for administration to patients (e.g., mammals including humans) either prophylactically or as part of a treatment regime.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A composition comprising:
a small molecule or pharmaceutically acceptable salt or solvate thereof, that alters the activity of or gene expression of a TMC protein in the testis.

2. The composition of claim 1 wherein the TMC protein is TMC5.

3. The composition of claim 1 wherein the small molecule is selected from the group consisting of: a TMC5 channel blocker, a TMC5 dimerization inhibitor, a TMC5-CIB1 interaction inhibitor, and combinations thereof.

4. The composition of claim 1, wherein the small molecule downregulates expression of the TMC protein in testis tissue.

5. The composition of claim 1, wherein the small molecule is a small interfering RNA targeting TMC5 mRNA.

6. The composition of claim 1, further comprising a pharmaceutically acceptable carrier.

7. The composition of claim 1 wherein the small molecule can cross the blood-testis barrier.

8. A method of diagnosing male infertility, comprising:
(1) quantitating expression level or activity of TMC5;
(2) comparing to a standard expression level; and,
(3) diagnosing infertility when the quantitated expression level is less than the standard expression level.

9. The method of claim 8, wherein the quantitating of expression level of TMC5 is performed on a biological sample selected from the group consisting of: semen, testicular tissue, and blood plasma.

10. The method of claim 8, wherein the quantitating of expression level of TMC5 comprises performing a nucleic acid amplification assay.

11. The method of claim 8, wherein the quantitating of activity of TMC5 comprises measuring calcium influx in isolated spermatogenic cells.

12. The method of claim 8, wherein the standard expression level is derived from a reference population of fertile male subjects.

13. The method of claim 8, further comprising: correlating the quantitated expression level of TMC5 with one or more semen quality parameters.

14. The method of claim 8, further comprising: repeating steps (1) through (3) after administering a TMC5-modulating agent to the subject.

15. A method of decreasing male fertility, comprising:
administering to a subject the composition according to claim 4.
